(19) United States
(12) Patent Application Publication
(10) Pub. No.: US 2019/0326020 A1
(43) Pub. Date: Oct. 24, 2019

(54) Video Conferencing and Virtual Appointments

(71) Applicant: Covidien LP, Mansfield, MA (US)

(72) Inventors: Jonathan James Woodward, Annapolis, MD (US); Amit Mukherjee, Elkridge, MD (US); Mark Kamensek, New Market, MD (US)

(21) Appl. No.: 15/957,316

(22) Filed: Apr. 19, 2018

(51) Int. Cl. G16H 80/00; H04N 7/15; H04N 7/14

(52) U.S. Cl. CPC G16H 80/00; H04N 7/147; H04N 7/15

(57) ABSTRACT

Methods, systems, and devices for initiating a video conference between a patient and a clinician are described. The method may include receiving an indication of an availability of the patient to participate in the video conference with the clinician, where the indication is based on detecting a proximity of the patient to a video conference device. The method may also determine an availability of the clinician to participate in the video conference with the patient. The method may further include initiating the video conference between the patient and the clinician, via the video conference device, based on the received indication of the availability of the patient and the determined availability of the clinician.

### BACKGROUND

The following relates generally to video conferencing and virtual appointments between patients and clinicians, and more specifically to initiating a video conference between a patient and a clinician.

In a healthcare facility such as a hospital, physiological parameters of the patient (e.g., heart rate, respiratory rate, blood pressure) may be monitored by one or more medical devices. The medical devices may be battery powered and may wirelessly transmit measured patient data over a wireless network within the hospital, thereby allowing the patient to move freely through the hospital while being monitored. Clinicians may remotely monitor the patient by accessing the patient data at a central nurse station or on any web enabled device connected to the network (e.g., smartphone or tablet).

The medical devices may be configured to transmit patient data over a network outside of the hospital, thereby allowing the patient to return home and continue patient care outside the hospital. In addition, the patient may conduct a video conference with a clinician as part of their at-home care plan. For example, the patient may manually set up a video conference between the patient and the clinician to answer questions or to perform follow-up diagnosis or consultations. However, a patient may be physically or mentally unable to manually set up a video conference.

### SUMMARY

The described features generally relate to methods, systems, devices, or apparatuses that support video conferencing and virtual appointments. A video conference server may receive an indication that a patient is available to participate in a video call (e.g., a video conference) with a clinician. The availability of the patient may be based on detecting the proximity of the patient to a video conference device (e.g., smartphone, computer, personal tablet, etc.). The video conference server may also determine an availability of the clinician to participate in the video call. Based on the availability of the clinician and the availability of the patient, the video conference server may initiate an impromptu video call between the patient and the clinician. In some cases, a time may be scheduled for the video call; however, the call may be initiated within a time threshold of the scheduled time if both the patient and clinician are available.

In some cases, the video call may be initiated based on a change in a previously diagnosed medical condition of the patient (e.g., increase in heart rate) or a detection of a new medical condition associated with the patient (e.g., elevated blood glucose levels for an extended period of time). The change in the medical condition may be detected by a live video feed of the patient, a motion sensor associated with patient, facial recognition of the patient, a medical sensor directly coupled with the patient, an audio sensor associated with patient, or by some combination of these sources.

A method for initiating a video conference between a patient and a clinician is described. The method may include receiving an indication of an availability of the patient to participate in the video conference with the clinician, wherein the indication is based at least in part on detecting a proximity of the patient to a video conference device, determining an availability of the clinician to participate in the video conference with the patient, and initiating the video conference between the patient and the clinician, via the video conference device, based at least in part on the received indication of the availability of the patient and the determined availability of the clinician.

Some examples of the method described herein may further include operations, features, means, or instructions for receiving an indication of a change in a medical condition associated with the patient, wherein the initiating the video conference between the patient and the clinician is further based at least in part on the received indication of the change in the medical condition. In some examples of the method described herein, the indication of the change in the medical condition may be based at least in part on a video feed of the patient, a motion detection of the patient, a facial recognition detection of the patient, a medical sensor coupled with the patient, an audio detection of the patient, or a combination thereof. In some examples, the change in the medical condition may comprise a fall event, a seizure, becoming unconsciousness, or a combination thereof. The method may further include adjusting a watch status associated with the patient based at least in part on the indication of the change in the medical condition.

Some examples of the method described herein may further include operations, features, means, or instructions for detecting a medical condition associated with the patient, wherein the initiating the video conference between the patient and the clinician is further based at least in part on the detected medical condition. Some examples of the method described herein may further include operations, features, means, or instructions for identifying a scheduled time for the video conference and determining a current time associated with the received indication of the availability of the patient, wherein the initiating the video conference is based at least in part on the current time being within a time threshold of the scheduled time.

In some examples of the method described herein, detecting the proximity of the patient to the video conference device may be based at least in part on Bluetooth signaling, motion detection, infrared detection, voice activation, facial recognition, or a combination thereof. Some examples of the method described herein may further include operations, features, means, or instructions for determining a connection status between a medical sensor coupled with the patient and the video conference device, wherein the detecting the proximity of the patient to the video conference device is further based at least in part on the determined connection status.

Some examples of the method described herein may further include operations, features, means, or instructions for transmitting an availability alert associated with the availability of the patient to the clinician. Some examples of the method described herein may further include operations, features, means, or instructions for receiving an indication of the availability of the clinician to participate in the video conference with the patient, wherein the determining the availability of the clinician is further based at least in part on the received indication of the availability of the clinician. In some examples of the method described herein, the indication may be based at least in part on detecting if the clinician is participating in a video conference with a second patient. Some examples of the method described herein may further include operations, features, means, or instructions for determining a relationship between the receiving the indication of the availability of the patient and the initiating the video conference between the patient and the clinician and storing the relationship between the receiving and the initiating.

### BRIEF DESCRIPTION OF THE DRAWINGS

FIG. 1 illustrates an example of a wireless patient monitoring system that supports video conferencing and virtual appointments in accordance with aspects of the present disclosure.

FIG. 2 illustrates a system for initiating a video conference between a patient and a clinician that supports video conferencing and virtual appointments in accordance with aspects of the present disclosure.

FIG. 3 illustrates an example process flow that supports video conferencing and virtual appointments in accordance with aspects of the present disclosure.

FIGS. 4 through 6 show block diagrams of a device that supports video conferencing and virtual appointments in accordance with aspects of the present disclosure.

FIG. 7 illustrates a block diagram of a system including a video conference server that supports video conferencing and virtual appointments in accordance with aspects of the present disclosure.

FIGS. 8 through 11 illustrate methods for video conferencing and virtual appointments in accordance with aspects of the present disclosure.

### DETAILED DESCRIPTION

In some home-based monitoring systems, a patient may return home from the healthcare facility with instructions for at-home patient care. If the patient returns home with questions regarding at-home treatment or concerns regarding a medical condition, the patient may initiate an impromptu video conference with the clinician. Video conferences may also be used for regular clinical diagnostic or consulting purposes. In accordance with aspects of the present disclosure, a video communication system may automatically schedule impromptu video consultations between the patient and the clinician. The video consultation may be based on an event to trigger the video call, an availability of the patient, an availability of the clinician, or a combination of these factors. The automatic scheduling and initiation of the video call may be beneficial for patients who are physically or mentally unable to set up a video conference.

In some cases, the event to trigger a call may include monitoring or detecting medical conditions associated with the patient. The medical conditions may be monitored or detected through a video feed, medical sensors associated with the patient, an audio device, or some combination of these sources. The video monitor may also monitor and detect events at the patient's home. In some cases, the video monitor may detect a triggering event that may automatically initiate a video consultation between the patient and the clinician. For example, a triggering event may be a seizure, a fall, or an increase in heart rate that may indicate a heart attack. Alternatively, if the video monitor detects a triggering event, the system may alert the clinician that a video consultation may be necessary before initiating the video conference. A watch status of the patient may also be elevated based on the triggering event and a severity of the medical condition detected.

The video communication system may also determine the availability for the participants involved with the video conference call. For example, the video communication system may determine if the patient is available for the video consultation (e.g., whether the patient is at home, in the room with the video monitor, etc.). The video communication system may also determine if the clinician is available for the video consultation (e.g., not with another patient, on call, near the video conference equipment, etc.). The availability may be detected based on the proximity of the patient, clinician, or both to the video monitor. For example, Bluetooth signaling or motion detection may determine the proximity of the patient and the clinician. In some cases, the availability may be detected by voice activation or by manual activation (e.g., direct access to the video monitor). The video communication system may send a notification (e.g., an availability alert) to the patient based on the availability of the clinician. Likewise, the video communication system may send a notification to the clinician based on the availability of the patient.

The time of the video consultation may be automatically initiated based on a change in the medical condition of the patient or a detection of a newly diagnosed medical condition. Alternatively, the video conference call may be scheduled within a predetermined time that the clinician may respond in order to prevent future complications associated with the medical condition of the patient. Therefore, an impromptu video consultation may be scheduled between the patient and the clinician to avoid increased risk to the patient.

Aspects of the disclosure are initially described in the context of a wireless patient monitoring system. Aspects of the disclosure are further illustrated by and described with reference to apparatus diagrams, system diagrams, and flowcharts that relate to video conferencing and virtual appointments.

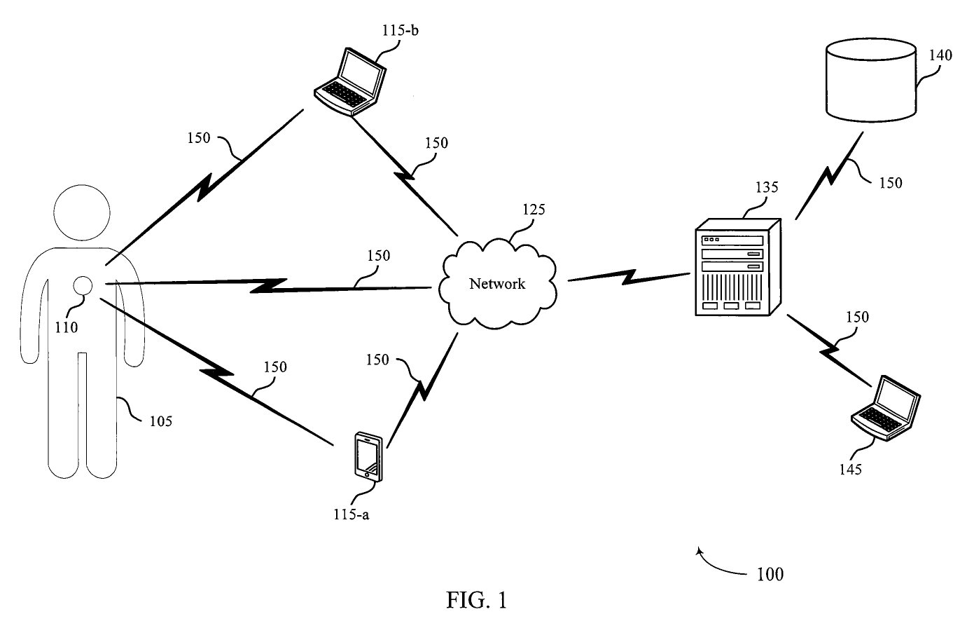

FIG. 1 illustrates an example of a wireless patient monitoring system 100 that supports video conferencing and virtual appointments in accordance with various embodiments of the present disclosure. The wireless patient monitoring system 100 may include a patient 105 wearing, carrying, or otherwise coupled with a medical device 110. Although a single medical device 110 is shown, multiple medical devices 110 may be coupled to the patient 105. The patient 105 may be a patient in a hospital, nursing home, home care, a medical facility, another care facility, or at a home residence. The medical device 110 may transmit signals via wireless communications links 150 to computing devices 115 or to a network 125.

The medical device 110 may include one or more sensors configured to collect a variety of physiological parameters as well as information related to the location and movement of the patient 105. For example, the medical device 110 may include a pulse oximetry (SpO2) sensor, a capnography sensor, a heart rate sensor, a blood pressure sensor, an electrocardiogram (ECG) sensor, a respiratory rate sensor, a glucose level sensor, a depth of consciousness sensor, a body temperature sensor, an accelerometer, a global positioning sensor, a sensor which triangulates position from multiple local computing devices 115, or any other sensor configured to collect physiological, location, or motion data associated with the patient 105.

The medical device 110 may be coupled with the patient 105 in a variety of ways depending on the data being collected. For example, the medical device 110 may be directly coupled with the patient 105 (e.g., physically connected to the patient's chest, worn around the patient's wrist, attached to the patient's finger, or positioned over the patients nose or mouth). The data collected by the medical device 110 may be wirelessly transmitted to either the computing devices 115 or to the remote computing device 145 (via the network 125 and central station 135). Data transmission may occur via, for example, frequencies appropriate for a personal area network (such as Bluetooth, Bluetooth Low Energy (BLE), or IR communications) or local (e.g., wireless local area network (WLAN)) or wide area network (WAN) frequencies such as radio frequencies specified by IEEE standards (e.g., IEEE 802.15.4 standard, IEEE 802.11 standard (Wi-Fi), IEEE 802.16 standard (WiMAX), etc.).

Computing device 115-a may be a wireless device such as a tablet, cellular phone, personal digital assistant (PDA), a dedicated receiver, or other similar device or a spatially distributed network of devices configured to receive signals from the medical device 110. Computing device 115-b may be a wireless laptop computer, a clinician Workstation on Wheels, or a smart hospital bed configured to receive signals from the medical device 110. Computing device 115-b may also be a video conference device configured receive an availability notification associated with patient 105 and initiate a video conference between patient 105 and a clinician. The computing devices 115 may be in communication with a central station 135 via network 125.

The medical device 110 may also communicate directly with the central station 135 via the network 125. The central station 135 may be a server or a central nurse station located within the hospital or in a remote location (e.g., at the home of patient 105). The central station 135 may be in further communication with one or more remote computing devices 145, thereby allowing a clinician to remotely monitor the patient 105. The central station 135 may also be in communication with various remote databases 140 where the collected patient data may be stored. In some cases, the remote databases 140 include electronic medical records (EMR) applications for storing and sharing patient data.

In accordance with various embodiments, methods and apparatuses are described for initiating a video conference between patient 105 and a clinician. When patient 105 returns home after an appointment with the clinician, patient 105 may be monitored based on a medical condition or patient 105 may have follow-up questions for the clinician based on their medical condition. In that case, a video conference server (e.g., central station 135) may receive an indication that patient 105 is available for a video conference based on the proximity of patient 105 to a video conference device (e.g., computing device 115). The video conference server may also determine the availability of the clinician to participate in the video conference.

Based on the availability of patient 105 and the clinician, the video conference server may initiate the video conference. The video conference may also be initiated based on the video conference server detecting a medical condition associated with patient 105 or detecting a change in the medical condition. For example, the video conference may initiated if the video conference server detects that patient 105 falls, becomes unconscious, or experiences a seizure.

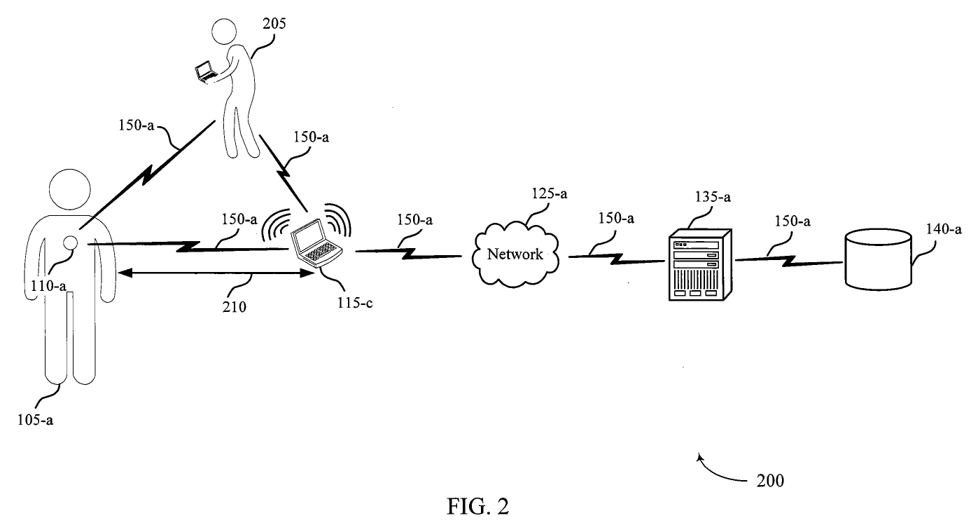

FIG. 2 illustrates a system 200 for initiating a video conference between a patient and a clinician that supports video conferencing and virtual appointments in accordance with various aspects of the present disclosure. System 200 may be an example of aspects of wireless patient monitoring system 100 and may include a patient 105-a wearing, carrying, or otherwise coupled with a medical device 110-a. System 200 may also be in communication with a clinician 205.

Patient 105-a may communicate bidirectionally via wired or wireless communication links 150-a to medical video device 115-c. Medical video device 115-c may be an example of aspects of computing device 115. Medical video device 115-c may also be an example of a device that receives an availability notification (e.g., indication of availability) from patient 105-a, clinician 205, or both. In some cases, medical video device 115-c may include a webcam configured to initiate a video conference between patient 105-a and clinician 205.

In some examples, medical video device 115-c may communicate bidirectionally via wired or wireless communication links 150-a to video conference server 135-a via network 125-a. Video conference server 135-a may also communicate bidirectionally via wired or wireless communication links 150-a to database 140-a to retrieve medical information. Video conference server 135-a may be an example of aspects of central station 135.

In some cases, video conference server 135-a may receive an availability notification associated with patient 105-a based on detecting a proximity of patient to medical video device 115-c (e.g., video conference device). The proximity of patient 105-a may be based on distance 210. For example, when patient 105-a is within distance 210, medical video device 115-c may activate (e.g., turn on) and detect the presence of patient 105-a. In some cases, the proximity may be detected based on Bluetooth signaling, motion detection of patient 105-a, or infrared detection. For example, video conference server 135-a may be able to detect the proximity of patient 105-a based on a wearable device associated with patient 105-a (e.g., smart watch, medical sensor 110-a, navigation on personal hand held device, etc.) In some cases, proximity detection based on Bluetooth signaling may not require medical video device 115-c to be in pairing mode with a device associated with patient 105-a. The proximity may also be detected based on voice activation associated with patient 105-a or facial recognition of patient 105-a. In some cases, medical sensor 110-a coupled with patient 105-a may establish a connection with medical video device 115-c, and video conference server 135-a may detect the proximity of patient 105-a based on the connection.

Clinician 205 may also communicate bidirectionally via wired or wireless communication links 150-a to medical video device 115-c. In some examples, video conference server 135-a may determine the availability of the clinician to participate in a video conference with patient 105-a. Based on the availability of clinician 205, video conference server 135-a may receive an availability notification (e.g., indication of availability) associated with clinician 205. In some examples, video conference server 135-a may transmit the availability notification to patient 105-a to initiate the video conference. In some cases, video conference server 135-a may refrain from transmitting the availability notification to patient 105-a if clinician 205 is in an appointment (e.g., a video conference) with another patient or is otherwise unavailable (e.g., not on duty). Clinician 205 may also receive an alert (e.g., availability alert) when patient 105-a is available for the video conference.

Video conference server 135-a may initiate a video conference between patient 105-a and clinician 205. For example, the video conference may be initiated after determining the availability of patient 105-a and clinician 205. In some cases, a change in a medical condition associated with patient 105-a may initiate the video conference. For example, if medical sensor 110-a associated with patient 105-a detects a sudden decrease in heart rate of patient 105-a, a video conference with clinician 205 may be initiated. That is, medical sensor 110-a coupled to patient 105-a may detect a change in the medical condition, and indicate the change to video conference server 135-a to initiate the video conference. Based on the detected change in the medical condition, video conference server 135-a may adjust a watch status for patient 105-a. For example, if video conference server 135-a detects more than one fall event associated with patient 105-a, video conference server 135-a may tag patient 105-a at risk for falling and increase the watch status.

In some examples, a live video feed or motion detection of patient 105-a may indicate a change in the medical condition associated with patient 105-a. For example, if patient 105-a falls and becomes unconscious, video conference server 135-a may detect the change in the medical condition of patient 105-a and initiate a video conference with clinician 205. In other examples, the detected change in medical condition may initiate a video conference call with an emergency response team.

In some cases, an audio detection of patient 105-a may indicate a change in medical condition associated with patient 105-a. For example, if patient 105-a verbally requests help or asks for assistance from an emergency help line after a fall, a video conference may be immediately initiated. In some examples, facial recognition of patient 105-a may initiate a video conference with clinician 205. For example, video conference server 135-a may detect if patient 105-a is in front of medical video device 115-c and initiate a video conference with clinician 205. That is, patient 105-a may have a scheduled appointment with clinician 205 but may be able to interact with medical video device 115-c to initiate the video conference. Therefore, video conference server 135-a may detect the availability of patient 105-a based on facial recognition and initiate the video conference.

In some cases, video conference server 135-a may detect a medical condition associated with patient 105-a and initiate the video conference with clinician 205. For example, medical sensor 110-a may detect an increase in blood glucose level for an extended period of time. Therefore, medical sensor 110-a may indicate the detected medical condition to video conference server 135-a and initiate a video conference between patient 105-a and clinician 205. During the video conference, clinician may diagnose patient 105-a based on the detected medical condition or schedule an appointment with patient 105-a to discuss the detected medical condition.

In some cases, video conference server 135-a may automatically initiate a video conference between patient 105-a and clinician 205 based on determining the availability of patient 105-a and clinician 205. In other examples, video conference server 135-a may initiate a video conference call between patient 105-a and clinician 205 after identifying a time (e.g., scheduled time) for the video conference. For example, video conference server 135-a may determine a time (e.g., current time) when patient 105-a becomes available, and initiate the video conference call before reaching the scheduled time.

In some examples, video conference server 135-a may determine a relationship between the availability of patient 105-a and initiating the video conference call between patient 105-a and clinician 205. Video conference may then store the relationship between the availability and the video conference initiation. For example, the relationship may be stored in database 140-a to log the medical history of patient 105-a. In some cases, video conference server 135-a may retrieve the stored relationship based on a request to review the medical history of patient 105-a. For example, patient 105-a may receive multiple video conferences and comment on the number of video conferences. In that case, clinician 205 or other users may retrieve the stored relationship to verify the reason the video conference was initiated. For example, the medical history may determine the video conference was initiated because video conference server 135-a detected the proximity of patient 105-a and a motion detection (e.g., a fall) associated with the medical condition.

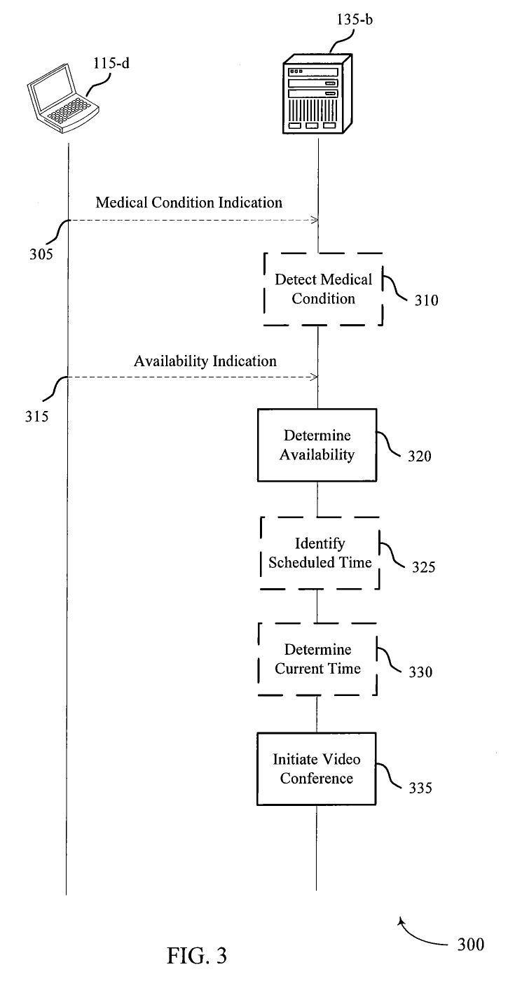

FIG. 3 illustrates an example process flow 300 that supports video conferencing and virtual appointments in accordance with various aspects of the present disclosure. Process flow 300 may include medical video device 115-d and video conference server 135-b, which may be respective examples of a computing device 115 and central station 135 as described with reference to FIGS. 1 and 2. Alternative examples of the following may be implemented, where some steps are performed in a different order or not at all. Some steps may additionally include additional features not mentioned above.

Video conference server 135-b may receive medical condition indication 305. Medical condition indication 305 may include an indication of a change in a medical condition (e.g., increase in heart rate, decrease in blood sugar, etc.) associated with the patient. In some examples, initiating a video conference between the patient and the clinician may be based on the received indication of the change in the medical condition. In other examples, medical condition indication 305 may be based on a video feed of the patient, a motion detection of the patient, a facial recognition detection of the patient, a medical sensor coupled with the patient, an audio detection of the patient, or a combination thereof. For example, medical condition indication 305 may include a fall event, a seizure, becoming unconsciousness, or a combination thereof. Based on the received medical condition indication 305, a watch status associated with the patient may be adjusted.

At block 310, video conference server 135-b may detect a medical condition associated with the patient or a change in a medical condition that may trigger initiation of a video conference. In some examples, initiating the video conference between the patient and the clinician may be based on the detected medical condition or change in medical condition.

Medical video device 115-d may transmit availability indication 315. Video conference server 135-b may receive availability indication 315, which may indicate the availability of the patient to participate in the video conference with the clinician. In some examples, availability indication 315 may be based on detecting a proximity of the patient to medical video device 115-d. The proximity of the patient may be detected by Bluetooth signaling, motion detection, infrared detection, voice activation, facial recognition, or a combination of these sources. In some cases, availability indication 315 may be an example of an availability alert associated with the availably of the patient. For example, availability indication 315 may be transmitted from the patient to the clinician.

In some examples, video conference server 135-b may receive availability indication 315 associated with the availability of the clinician. For example, determining the availability of the clinician may be based on the received availability indication 315 of the clinician. In some cases, availability indication 315 may be based on detecting if the clinician is participating in a video conference with a second patient (e.g., if the clinician is in another appointment).

At block 320, video conference server 135-b may determine an availability of the clinician to participate in the video conference with the patient. In some examples, video conference server 135-b may determine a connection status (e.g., connection) between a medical sensor coupled with the patient and medical video device 115-d, where detecting the proximity of the patient to the video conference device may be based on the determined connection status.

At block 325, video conference server 135-b may identify a scheduled time for the video conference. At block 330, video conference server 135-b may determine a current time associated with the received indication of the availability of the patient. In some cases, initiating the video conference may be based on the current time being within a time threshold of the scheduled time.

At block 335, video conference server 135-b may initiate the video conference between the patient and the clinician, via medical video device 115-d. In some examples, initiating the video conference may be based on availability indication 305 associated with the patient and the determined availability of the clinician. Video conference server 135-b may determine a relationship between availability indication 305 associated with the patient and the initiating the video conference between the patient and the clinician and store the relation between the receiving and the initiating (e.g., logging medical records for medical history related purposes).

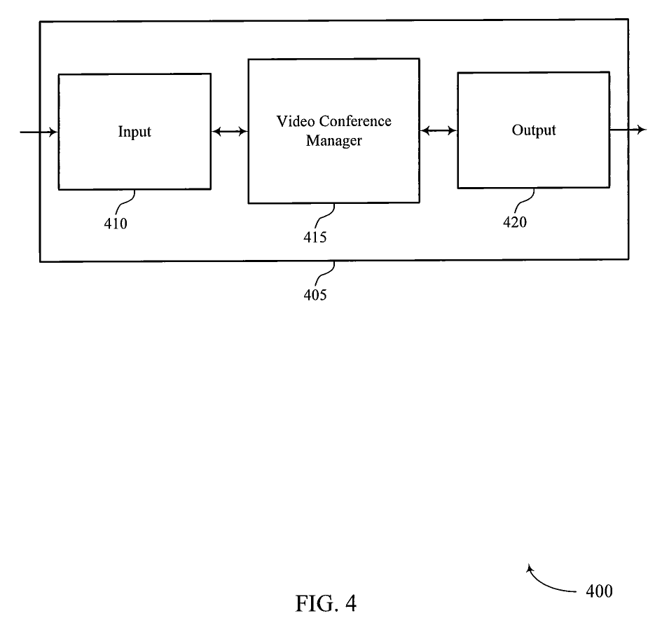

FIG. 4 shows a block diagram 400 of a device 405 that supports video conferencing and virtual appointments in accordance with aspects of the present disclosure. Device 405 may be an example of aspects of a video conference server as described herein. Device 405 may include input 410, video conference manager 415, and output 420. Device 405 may also include a processor. Each of these components may be in communication with one another (e.g., via one or more buses).

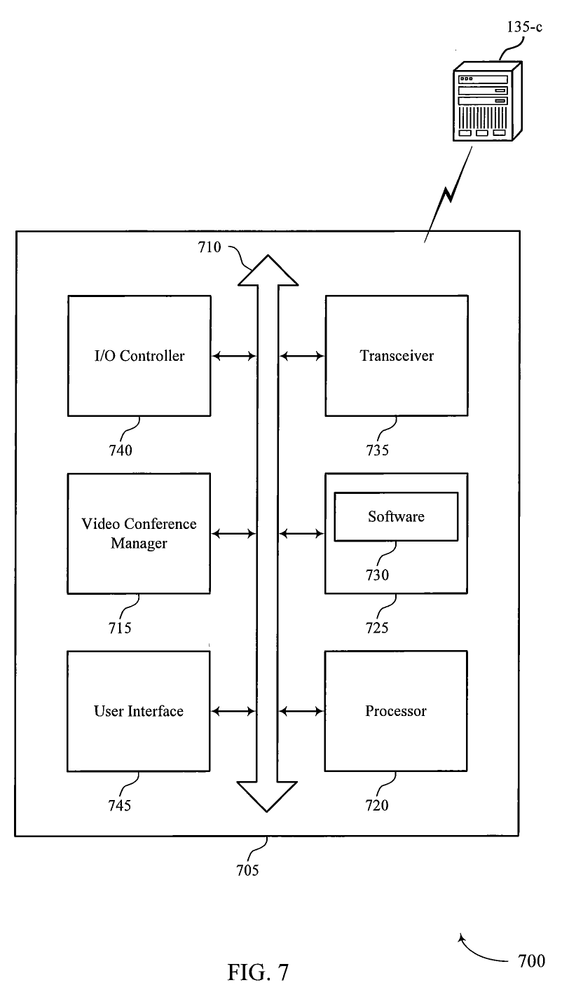

Video conference manager 415 may be an example of aspects of the video conference manager 715 described with reference to FIG. 7.

Video conference manager 415 and/or at least some of its various sub-components may be implemented in hardware, software executed by a processor, firmware, or any combination thereof. If implemented in software executed by a processor, the functions of the video conference manager 415 and/or at least some of its various sub-components may be executed by a general-purpose processor, a digital signal processor (DSP), an application-specific integrated circuit (ASIC), an field-programmable gate array (FPGA) or other programmable logic device, discrete gate or transistor logic, discrete hardware components, or any combination thereof designed to perform the functions described in the present disclosure. The video conference manager 415 and/or at least some of its various sub-components may be physically located at various positions, including being distributed such that portions of functions are implemented at different physical locations by one or more physical devices. In some examples, video conference manager 415 and/or at least some of its various sub-components may be a separate and distinct component in accordance with various aspects of the present disclosure. In other examples, video conference manager 415 and/or at least some of its various sub-components may be combined with one or more other hardware components, including but not limited to an I/O component, a transceiver, a network server, another computing device, one or more other components described in the present disclosure, or a combination thereof in accordance with various aspects of the present disclosure.

Video conference manager 415 may receive an indication of an availability of the patient to participate in the video conference with the clinician, where the indication is based on detecting a proximity of the patient to a video conference device, determine an availability of the clinician to participate in the video conference with the patient, and initiate the video conference between the patient and the clinician, via the video conference device, based on the received indication of the availability of the patient and the determined availability of the clinician.

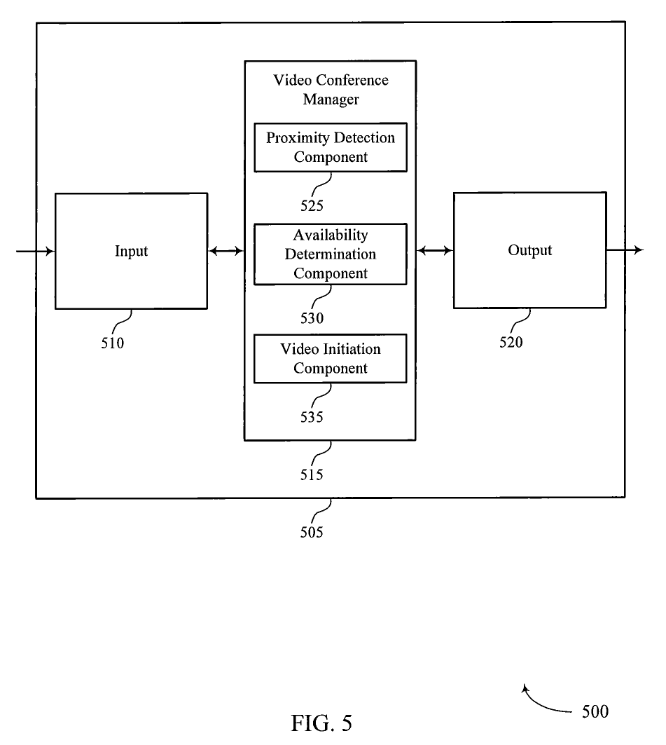

FIG. 5 shows a block diagram 500 of a device 505 that supports video conferencing and virtual appointments in accordance with aspects of the present disclosure. Device 505 may be an example of aspects of a device 405 or a video conference server as described with reference to FIG. 4. Device 505 may include input 510, video conference manager 515, and output 520. Device 505 may also include a processor. Each of these components may be in communication with one another (e.g., via one or more buses).

Video conference manager 515 may be an example of aspects of the video conference manager 715 described with reference to FIG. 7.

Video conference manager 515 may also include proximity detection component 525, availability determination component 530, and video initiation component 535.

Proximity detection component 525 may receive an indication of an availability of the patient to participate in the video conference with the clinician, where the indication is based on detecting a proximity of the patient to a video conference device. Proximity detection component 525 may determine a connection status between a medical sensor coupled with the patient and the video conference device, where the detecting the proximity of the patient to the video conference device is further based on the determined connection status. In some cases, the detecting the proximity of the patient to the video conference device is based on Bluetooth signaling, motion detection, infrared detection, voice activation, facial recognition, or a combination thereof.

Availability determination component 530 may determine an availability of the clinician to participate in the video conference with the patient, transmit an availability alert associated with the availability of the patient to the clinician, and receive an indication of the availability of the clinician to participate in the video conference with the patient, where the determining the availability of the clinician is further based on the received indication of the availability of the clinician. In some cases, the indication is based on detecting if the clinician is participating in a video conference with a second patient.

Video initiation component 535 may initiate the video conference between the patient and the clinician, via the video conference device, based on the received indication of the availability of the patient and the determined availability of the clinician.

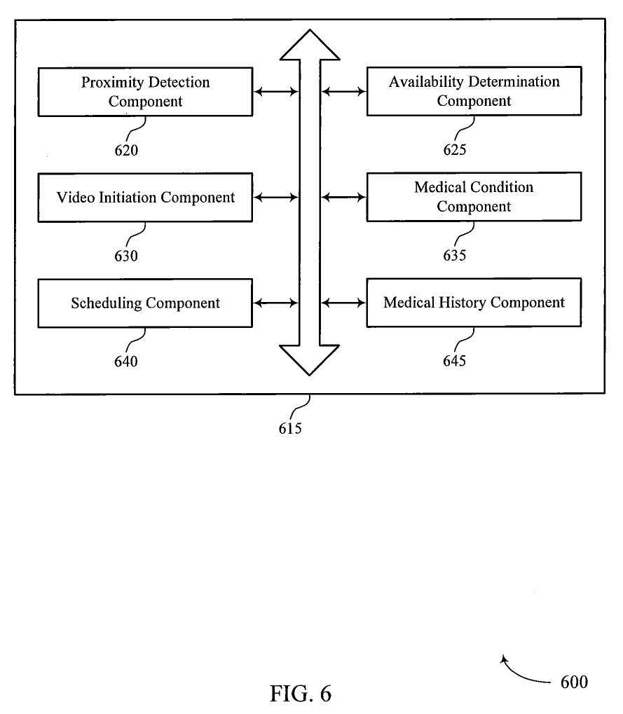

FIG. 6 shows a block diagram 600 of a video conference manager 615 that supports video conferencing and virtual appointments in accordance with aspects of the present disclosure. The video conference manager 615 may be an example of aspects of a video conference manager 415, a video conference manager 515, or a video conference manager 715 described with reference to FIGS. 4, 5, and 7. The video conference manager 615 may include proximity detection component 620, availability determination component 625, video initiation component 630, medical condition component 635, scheduling component 640, and medical history component 645. Each of these modules may communicate, directly or indirectly, with one another (e.g., via one or more buses).

Proximity detection component 620 may receive an indication of an availability of the patient to participate in the video conference with the clinician, where the indication is based on detecting a proximity of the patient to a video conference device. Proximity detection component 620 may determine a connection status between a medical sensor coupled with the patient and the video conference device, where the detecting the proximity of the patient to the video conference device is further based on the determined connection status. In some cases, the detecting the proximity of the patient to the video conference device is based on Bluetooth signaling, motion detection, infrared detection, voice activation, facial recognition, or a combination thereof.

Availability determination component 625 may determine an availability of the clinician to participate in the video conference with the patient, transmit an availability alert associated with the availability of the patient to the clinician, and receive an indication of the availability of the clinician to participate in the video conference with the patient, where the determining the availability of the clinician is further based on the received indication of the availability of the clinician. In some cases, the indication is based on detecting if the clinician is participating in a video conference with a second patient.

Video initiation component 630 may initiate the video conference between the patient and the clinician, via the video conference device, based on the received indication of the availability of the patient and the determined availability of the clinician.

Medical condition component 635 may receive an indication of a change in a medical condition associated with the patient, where the initiating the video conference between the patient and the clinician is further based on the received indication of the change in the medical condition. In some cases, medical condition component 635 may adjust a watch status associated with the patient based on the indication of the change in the medical condition. Medical condition component 635 may also detect a medical condition associated with the patient, where the initiating the video conference between the patient and the clinician is further based on the detected medical condition. In some cases, the indication of the change in the medical condition is based on a video feed of the patient, a motion detection of the patient, a facial recognition detection of the patient, a medical sensor coupled with the patient, an audio detection of the patient, or a combination thereof. In some cases, the change in the medical condition includes a fall event, a seizure, becoming unconsciousness, or a combination thereof.

Scheduling component 640 may identify a scheduled time for the video conference and determine a current time associated with the received indication of the availability of the patient, where the initiating the video conference is based on the current time being within a time threshold of the scheduled time.

Medical history component 645 may determine a relationship between the receiving the indication of the availability of the patient and the initiating the video conference between the patient and the clinician and store the relationship between the receiving and the initiating.

FIG. 7 shows a diagram of a system 700 including a device 705 that supports video conferencing and virtual appointments in accordance with aspects of the present disclosure. Device 705 may be an example of or include the components of device 405, device 505, or a video conference server as described above, e.g., with reference to FIGS. 4 and 5. Device 705 may include components for bi-directional voice and data communications including components for transmitting and receiving communications, including video conference manager 715, processor 720, memory 725, software 730, transceiver 735, I/O controller 740, and user interface 745. These components may be in electronic communication via one or more buses (e.g., bus 710).

Processor 720 may include an intelligent hardware device, (e.g., a general-purpose processor, a DSP, a central processing unit (CPU), a microcontroller, an ASIC, an FPGA, a programmable logic device, a discrete gate or transistor logic component, a discrete hardware component, or any combination thereof). In some cases, processor 720 may be configured to operate a memory array using a memory controller. In other cases, a memory controller may be integrated into processor 720. Processor 720 may be configured to execute computer-readable instructions stored in a memory to perform various functions (e.g., functions or tasks supporting video conferencing and virtual appointments).

Memory 725 may include random access memory (RAM) and read only memory (ROM). The memory 725 may store computer-readable, computer-executable software 730 including instructions that, when executed, cause the processor to perform various functions described herein. In some cases, the memory 725 may contain, among other things, a basic input/output system (BIOS) which may control basic hardware or software operation such as the interaction with peripheral components or devices.

Software 730 may include code to implement aspects of the present disclosure, including code to support video conferencing and virtual appointments. Software 730 may be stored in a non-transitory computer-readable medium such as system memory or other memory. In some cases, the software 730 may not be directly executable by the processor but may cause a computer (e.g., when compiled and executed) to perform functions described herein.

Transceiver 735 may communicate bi-directionally, via one or more antennas, wired, or wireless links as described above. For example, the transceiver 735 may represent a wireless transceiver and may communicate bi-directionally with another wireless transceiver. The transceiver 735 may also include a modem to modulate the packets and provide the modulated packets to the antennas for transmission, and to demodulate packets received from the antennas.

I/O controller 740 may manage input and output signals for device 705. I/O controller 740 may also manage peripherals not integrated into device 705. In some cases, I/O controller 740 may represent a physical connection or port to an external peripheral. In some cases, I/O controller 740 may utilize an operating system such as iOS®, ANDROID®, MS-DOS®, MS-WINDOWS®, OS/2®, UNIX®, LINUX®, or another known operating system. In other cases, I/O controller 740 may represent or interact with a modem, a keyboard, a mouse, a touchscreen, or a similar device. In some cases, I/O controller 740 may be implemented as part of a processor. In some cases, a user may interact with device 705 via I/O controller 740 or via hardware components controlled by I/O controller 740.

User interface 745 may enable a user to interact with device 705. In some embodiments, the user interface module 745 may include an audio device, such as an external speaker system, an external display device such as a display screen, or an input device (e.g., remote control device interfaced with the user interface module 745 directly or through the I/O controller module).

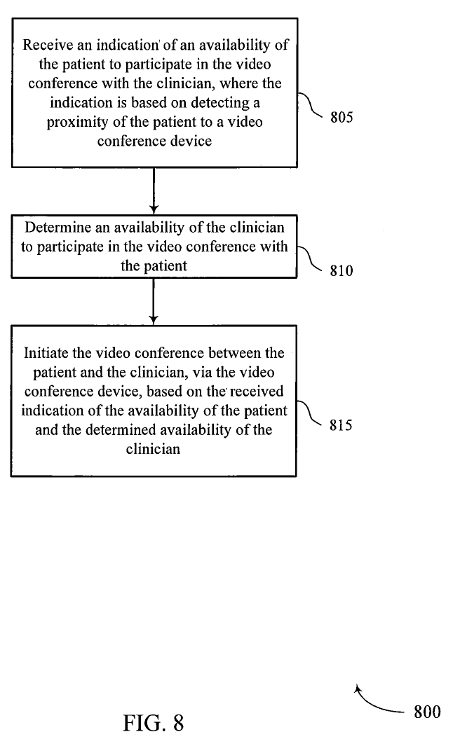

FIG. 8 shows a flowchart illustrating a method 800 for video conferencing and virtual appointments in accordance with aspects of the present disclosure. The operations of method 800 may be implemented by a video conference server or its components as described herein. For example, the operations of method 800 may be performed by a video conference manager as described with reference to FIGS. 4 through 7. In some examples, a video conference server may execute a set of codes to control the functional elements of the device to perform the functions described below. Additionally or alternatively, the video conference server may perform aspects of the functions described below using special-purpose hardware.

At 805 the video conference server may receive an indication of an availability of the patient to participate in the video conference with the clinician, wherein the indication is based at least in part on detecting a proximity of the patient to a video conference device. The operations of 805 may be performed according to the methods described herein. In certain examples, aspects of the operations of 805 may be performed by a proximity detection component as described with reference to FIGS. 4 through 7.

At 810 the video conference server may determine an availability of the clinician to participate in the video conference with the patient. The operations of 810 may be performed according to the methods described herein. In certain examples, aspects of the operations of 810 may be performed by an availability determination component as described with reference to FIGS. 4 through 7.

At 815 the video conference server may initiate the video conference between the patient and the clinician, via the video conference device, based at least in part on the received indication of the availability of the patient and the determined availability of the clinician. The operations of 815 may be performed according to the methods described herein. In certain examples, aspects of the operations of 815 may be performed by a video initiation component as described with reference to FIGS. 4 through 7.

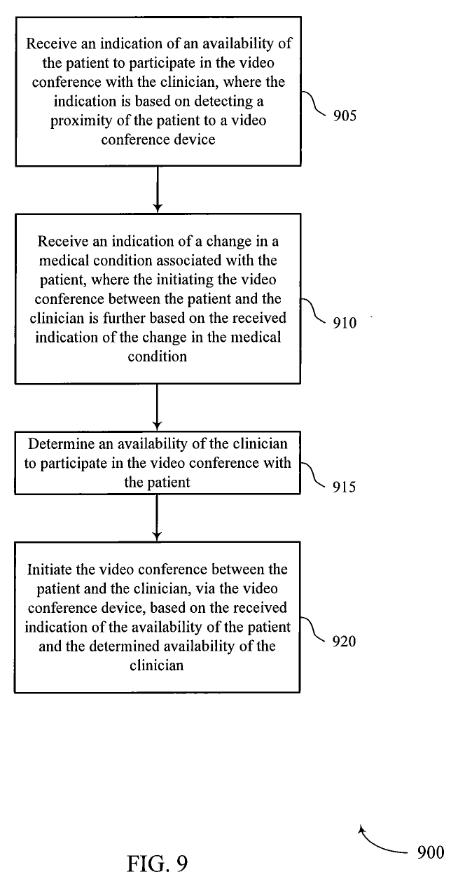

FIG. 9 shows a flowchart illustrating a method 900 for video conferencing and virtual appointments in accordance with aspects of the present disclosure. The operations of method 900 may be implemented by a video conference server or its components as described herein. For example, the operations of method 900 may be performed by a video conference manager as described with reference to FIGS. 4 through 7. In some examples, a video conference server may execute a set of codes to control the functional elements of the device to perform the functions described below. Additionally or alternatively, the video conference server may perform aspects of the functions described below using special-purpose hardware.

At 905 the video conference server may receive an indication of an availability of the patient to participate in the video conference with the clinician, wherein the indication is based at least in part on detecting a proximity of the patient to a video conference device. The operations of 905 may be performed according to the methods described herein. In certain examples, aspects of the operations of 905 may be performed by a proximity detection component as described with reference to FIGS. 4 through 7.

At 910 the video conference server may receive an indication of a change in a medical condition associated with the patient, wherein the initiating the video conference between the patient and the clinician is further based at least in part on the received indication of the change in the medical condition. The operations of 910 may be performed according to the methods described herein. In certain examples, aspects of the operations of 910 may be performed by a medical condition component as described with reference to FIGS. 4 through 7.

At 915 the video conference server may determine an availability of the clinician to participate in the video conference with the patient. The operations of 915 may be performed according to the methods described herein. In certain examples, aspects of the operations of 915 may be performed by an availability determination component as described with reference to FIGS. 4 through 7.

At 920 the video conference server may initiate the video conference between the patient and the clinician, via the video conference device, based at least in part on the received indication of the availability of the patient and the determined availability of the clinician. The operations of 920 may be performed according to the methods described herein. In certain examples, aspects of the operations of 920 may be performed by a video initiation component as described with reference to FIGS. 4 through 7.

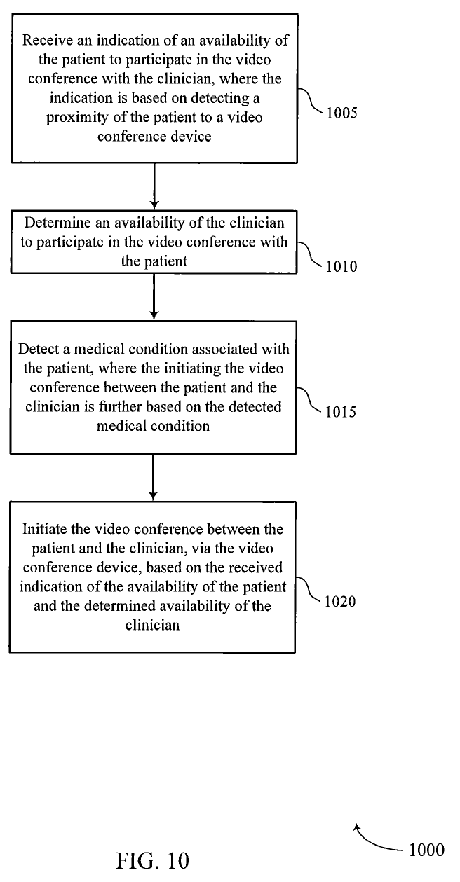

FIG. 10 shows a flowchart illustrating a method 1000 for video conferencing and virtual appointments in accordance with aspects of the present disclosure. The operations of method 1000 may be implemented by a video conference server or its components as described herein. For example, the operations of method 1000 may be performed by a video conference manager as described with reference to FIGS. 4 through 7. In some examples, a video conference server may execute a set of codes to control the functional elements of the device to perform the functions described below. Additionally or alternatively, the video conference server may perform aspects of the functions described below using special-purpose hardware.

At 1005 the video conference server may receive an indication of an availability of the patient to participate in the video conference with the clinician, wherein the indication is based at least in part on detecting a proximity of the patient to a video conference device. The operations of 1005 may be performed according to the methods described herein. In certain examples, aspects of the operations of 1005 may be performed by a proximity detection component as described with reference to FIGS. 4 through 7.

At 1010 the video conference server may determine an availability of the clinician to participate in the video conference with the patient. The operations of 1010 may be performed according to the methods described herein. In certain examples, aspects of the operations of 1010 may be performed by an availability determination component as described with reference to FIGS. 4 through 7.

At 1015 the video conference server may detect a medical condition associated with the patient, wherein the initiating the video conference between the patient and the clinician is further based at least in part on the detected medical condition. The operations of 1015 may be performed according to the methods described herein. In certain examples, aspects of the operations of 1015 may be performed by a medical condition component as described with reference to FIGS. 4 through 7.

At 1020 the video conference server may initiate the video conference between the patient and the clinician, via the video conference device, based at least in part on the received indication of the availability of the patient and the determined availability of the clinician. The operations of 1020 may be performed according to the methods described herein. In certain examples, aspects of the operations of 1020 may be performed by a video initiation component as described with reference to FIGS. 4 through 7.

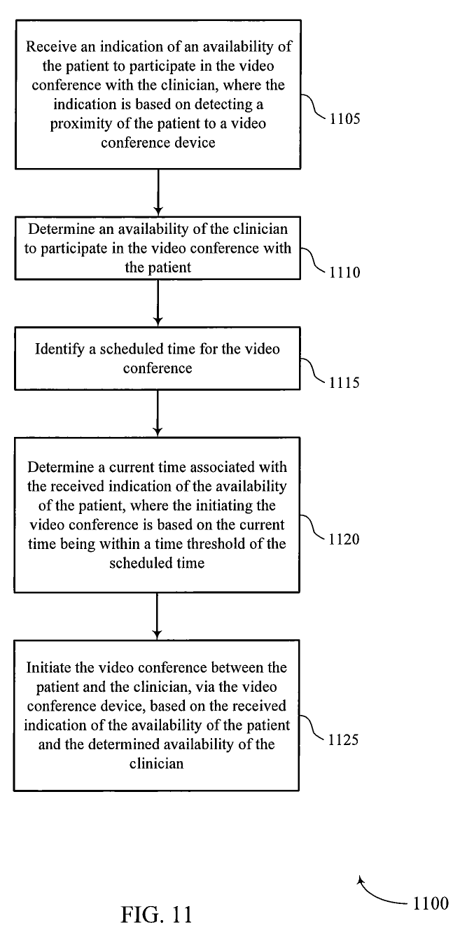

FIG. 11 shows a flowchart illustrating a method 1100 for video conferencing and virtual appointments in accordance with aspects of the present disclosure. The operations of method 1100 may be implemented by a video conference server or its components as described herein. For example, the operations of method 1100 may be performed by a video conference manager as described with reference to FIGS. 4 through 7. In some examples, a video conference server may execute a set of codes to control the functional elements of the device to perform the functions described below. Additionally or alternatively, the video conference server may perform aspects of the functions described below using special-purpose hardware.

At 1105 the video conference server may receive an indication of an availability of the patient to participate in the video conference with the clinician, wherein the indication is based at least in part on detecting a proximity of the patient to a video conference device. The operations of 1105 may be performed according to the methods described herein. In certain examples, aspects of the operations of 1105 may be performed by a proximity detection component as described with reference to FIGS. 4 through 7.

At 1110 the video conference server may determine an availability of the clinician to participate in the video conference with the patient. The operations of 1110 may be performed according to the methods described herein. In certain examples, aspects of the operations of 1110 may be performed by an availability determination component as described with reference to FIGS. 4 through 7.

At 1115 the video conference server may identify a scheduled time for the video conference. The operations of 1115 may be performed according to the methods described herein. In certain examples, aspects of the operations of 1115 may be performed by a scheduling component as described with reference to FIGS. 4 through 7.

At 1120 the video conference server may determine a current time associated with the received indication of the availability of the patient, wherein the initiating the video conference is based at least in part on the current time being within a time threshold of the scheduled time. The operations of 1120 may be performed according to the methods described herein. In certain examples, aspects of the operations of 1120 may be performed by a scheduling component as described with reference to FIGS. 4 through 7.

At 1125 the video conference server may initiate the video conference between the patient and the clinician, via the video conference device, based at least in part on the received indication of the availability of the patient and the determined availability of the clinician. The operations of 1125 may be performed according to the methods described herein. In certain examples, aspects of the operations of 1125 may be performed by a video initiation component as described with reference to FIGS. 4 through 7.

It should be noted that the methods described above describe possible implementations, and that the operations and the steps may be rearranged or otherwise modified and that other implementations are possible. Furthermore, aspects from two or more of the methods may be combined.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

Information and signals described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a digital signal processor (DSP), an ASIC, an field programmable gate array (FPGA) or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration). A processor may in some cases be in electronic communication with a memory, where the memory stores instructions that are executable by the processor. Thus, the functions described herein may be performed by one or more other processing units (or cores), on at least one integrated circuit (IC). In various examples, different types of ICs may be used (e.g., Structured/Platform ASICs, an FPGA, or another semi-custom IC), which may be programmed in any manner known in the art. The functions of each unit may also be implemented, in whole or in part, with instructions embodied in a memory, formatted to be executed by one or more general or application-specific processors.

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described above may be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

Computer-readable media includes both non-transitory computer storage media and communication media including any medium that facilitates transfer of a computer program from one place to another. A non-transitory storage medium may be any available medium that can be accessed by a general purpose or special purpose computer. By way of example, and not limitation, non-transitory computer-readable media may comprise RAM, ROM, electrically erasable programmable read only memory (EEPROM), compact disk (CD) ROM or other optical disk storage, magnetic disk storage or other magnetic storage devices, or any other non-transitory medium that may be used to carry or store desired program code means in the form of instructions or data structures and that may be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, digital subscriber line (DSL), or wireless technologies such as infrared, radio, and microwave are included in the definition of medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

What is claimed is:

1. A method for initiating a video conference between a patient and a clinician, comprising:
receiving an indication of an availability of the patient to participate in the video conference with the clinician, wherein the indication is based at least in part on detecting a proximity of the patient to a video conference device;
determining an availability of the clinician to participate in the video conference with the patient; and
initiating the video conference between the patient and the clinician, via the video conference device, based at least in part on the received indication of the availability of the patient and the determined availability of the clinician.

2. The method of claim 1, further comprising:
receiving an indication of a change in a medical condition associated with the patient, wherein the initiating the video conference between the patient and the clinician is further based at least in part on the received indication of the change in the medical condition.

3. The method of claim 2, wherein the indication of the change in the medical condition is based at least in part on a video feed of the patient, a motion detection of the patient, a facial recognition detection of the patient, a medical sensor coupled with the patient, an audio detection of the patient, or a combination thereof.

4. The method of claim 2, wherein the change in the medical condition comprises a fall event, a seizure, becoming unconsciousness, or a combination thereof.

5. The method of claim 2, further comprising:
adjusting a watch status associated with the patient based at least in part on the indication of the change in the medical condition.

6. The method of claim 1, further comprising:
detecting a medical condition associated with the patient, wherein the initiating the video conference between the patient and the clinician is further based at least in part on the detected medical condition.

7. The method of claim 1, further comprising:
identifying a scheduled time for the video conference; and
determining a current time associated with the received indication of the availability of the patient, wherein the initiating the video conference is based at least in part on the current time being within a time threshold of the scheduled time.

8. The method of claim 1, wherein the detecting the proximity of the patient to the video conference device is based at least in part on Bluetooth signaling, motion detection, infrared detection, voice activation, facial recognition, or a combination thereof.

9. The method of claim 1, further comprising:
determining a connection status between a medical sensor coupled with the patient and the video conference device, wherein the detecting the proximity of the patient to the video conference device is further based at least in part on the determined connection status.

10. The method of claim 1, further comprising:
transmitting an availability alert associated with the availability of the patient to the clinician.

11. The method of claim 1, further comprising:
receiving an indication of the availability of the clinician to participate in the video conference with the patient, wherein the determining the availability of the clinician is further based at least in part on the received indication of the availability of the clinician.

12. The method of claim 11, wherein the indication is based at least in part on detecting if the clinician is participating in a video conference with a second patient.

13. The method of claim 1, further comprising:
determining a relationship between the receiving the indication of the availability of the patient and the initiating the video conference between the patient and the clinician; and
storing the relationship between the receiving and the initiating.

14. An apparatus for initiating a video conference between a patient and a clinician, comprising:
a processor;
memory in electronic communication with the processor; and
instructions stored in the memory and executable by the processor to cause the apparatus to:
receive an indication of an availability of the patient to participate in the video conference with the clinician, wherein the indication is based at least in part on detecting a proximity of the patient to a video conference device;
determine an availability of the clinician to participate in the video conference with the patient; and
initiate the video conference between the patient and the clinician, via the video conference device, based at least in part on the received indication of the availability of the patient and the determined availability of the clinician.

15. The apparatus of claim 14, wherein the instructions are further executable by the processor to cause the apparatus to:
receive an indication of a change in a medical condition associated with the patient, wherein the initiating the video conference between the patient and the clinician is further based at least in part on the received indication of the change in the medical condition.

16. The apparatus of claim 15, wherein the indication of the change in the medical condition is based at least in part on a video feed of the patient, a motion detection of the patient, a facial recognition detection of the patient, a medical sensor coupled with the patient, an audio detection of the patient, or a combination thereof.

17. The apparatus of claim 15, wherein the change in the medical condition comprises a fall event, a seizure, becoming unconsciousness, or a combination thereof.

18. The apparatus of claim 14, wherein the instructions are further executable by the processor to cause the apparatus to:
detect a medical condition associated with the patient, wherein the initiating the video conference between the patient and the clinician is further based at least in part on the detected medical condition.

19. The apparatus of claim 14, wherein the instructions are further executable by the processor to cause the apparatus to:
identify a scheduled time for the video conference; and
determine a current time associated with the received indication of the availability of the patient, wherein the initiating the video conference is based at least in part on the current time being within a time threshold of the scheduled time.

20. A non-transitory computer readable medium storing code for initiating a video conference between a patient and a clinician, the code comprising instructions executable by a processor to:
receive an indication of an availability of the patient to participate in the video conference with the clinician, wherein the indication is based at least in part on detecting a proximity of the patient to a video conference device;
determine an availability of the clinician to participate in the video conference with the patient; and
initiate the video conference between the patient and the clinician, via the video conference device, based at least in part on the received indication of the availability of the patient and the determined availability of the clinician.
